(19) United States
(12) Patent Application Publication
(10) Pub. No.: US 2025/0143813 A1
(43) Pub. Date: May 8, 2025

(54) ROBOTIC ACTIVE AND DYNAMIC COLLISION AVOIDANCE SYSTEM AND METHOD

(71) Applicant: LEM Surgical AG, Bern (CH)

(72) Inventor: Yossi BAR, Muri bei Bern (CH)

(73) Assignee: LEM Surgical AG, Bern (CH)

(21) Appl. No.: 18/969,084

(22) Filed: Dec. 4, 2024

Related U.S. Application Data
(63) Continuation-in-part of application No. PCT/IB2022/058988, filed on Sep. 22, 2022.
(60) Provisional application No. 63/349,146, filed on Jun. 6, 2022.
(60) Provisional application No. 63/609,490, filed on Dec. 13, 2023.

(51) Int. Cl. A61B 34/30; A61B 90/00

(52) U.S. Cl. CPC A61B 34/30; A61B 90/361; A61B 2034/306

(57) ABSTRACT

Systems and methods for preventing collisions between robotic arms of a multi-arm surgical robotic systems and objects in a surgical field in are described. The positions and motions of the surgical robotic are tracked kinematically while the positions of the objects are tracked by a camera or other sensor held on a surveillance arm of the surgical robot. Each of the robotic surgical arms and the surveillance arm is mounted on a common chassis or cart to define a single surgical space coordinate system to facilitate tracking.

### CROSS-REFERENCE TO RELATED APPLICATIONS

This application is a continuation-in-part of PCT/IB2022/058988, filed Sep. 22, 2022, which claims the benefit of U.S. Provisional No. 63/349,146, filed Jun. 6, 2022; this application also claims the benefit of U.S. Provisional No. 63/609,490, filed Dec. 13, 2023, the full disclosures of which are incorporated herein by reference.

### BACKGROUND

This application relates generally to systems and methods for avoiding collisions in a surgical robotic system. More particularly, the application relates to systems and methods for preventing robotic arms from colliding with other robotic arms, surgical tools, surgical personnel, and the patient.

Collision avoidance is of particular concern in surgical robot systems where multiple robot arms are manipulated in close proximity to each other as well as to surgical tools and other equipment, to the patient, and to surgical personnel. Tracking and avoiding unintended interactions of the surgical robot arms is made more difficult by the constantly changing environment. Unlike most industrial robots where arm movements are predictable and repetitive, surgical robots must accommodate unpredictable changes in patient position, target anatomy, and movement of the personnel in the surgical field where the robotic arms are deployed.

A common solution to this problem has been the use of collaborative robots (“cobots”) to minimize unintended collisions and other interactions of the surgical robot arms. Cobots as designed to detect collisions with a human or other object and to stop the arm after the collision is detected. While helpful, even the best cobots have a detection threshold of one- to two-kilogram force which is not acceptable in a surgical environment where even a small collision force below one kilogram could injure the patient and/or the medical staff and in some cases could even be lethal to the patient, e.g., where a robotic arm or surgeon is operating on the patient with a scalpel or other sharp tool.

Surgical robotic systems which track the positions of robotic arms in real time, for example using cameras or other sensors, can employ collision avoidance algorithms to detect and predict possible collision to prevent collisions before they occur. This is a significant improvement over the cobots which stop the arms only after a collision is detected. Such systems, however, are often designed for single-arm robotic systems and are not able to possess all information useful in predicting collisions, e.g., position, trajectory. arm speed and the like for al arms in a multiple-arm system as well as tracking the patient anatomy and the locations of the hands and arms of the surgical personnel.

For these reasons, it would be desirable to provide improved and alternative collision avoidance systems for use with surgical robotic systems. Such collision avoidance systems should be configured to track and control the movement of surgical robotic arms, usually multiple surgical robotic arms, in a surgical field. Where the arms are in dynamic movement in the presence of the patient and surgical personnel. The collision avoidance systems should be able to continuously track unpredictable and random changes in the surgical field and provide this information to a controller which manages movements of the robotic arms relative to each other as well as the patient anatomy and the surgical personnel to reduce the risk of, and preferably fully prevent, unintended collision and other interactions from happening before they occur. At least some of these objectives will be met by the inventions described and claimed herein.

### SUMMARY

The systems, apparatus, and methods disclosed and claimed herein are configured to reduce and preferably eliminate the risk of surgical robotic arms and their tools and end effectors colliding or unintentionally interacting with other objects in the surgical space, including but not limited to other surgical robotic arms and their tools and end effectors, free tools and surgical equipment not being controlled by the surgical robot, surgical personnel, and patient anatomy not involved in the surgical procedure. While useful with a wide variety of surgical robots, the collision avoidance systems of the present invention are particularly intended for use with spinal and other orthopedic surgical robots and robotic systems where multiple robotic arms are operating in close proximity to each other as well as devices such as cameras, surgical tools, end effectors, and equipment.

In exemplary embodiments, the collision avoidance systems of the present invention are employed with mobile, multiple-armed surgical robots system comprising, consisting essentially of, or consisting or a single chassis or cart or multiple chasses or carts which may be mechanically connected to define a single surgical space (or coordinate space) in which the multiple robotic arms may be kinematically controlled by a common controller. The surgical robot will usually also comprise one or more cameras or other sensors carried by one or more robotic arms which are also mounted on the common chassis or cart and controlled by the common controller.

The common controller will deploy the surgical robotic arms around a patient during surgery. The patient's position, anatomy and “surface topology” are monitored and tracked by the cameras or other sensors as they continuously change during the surgery. The cameras or other sensors can also track surgical tools, such as forceps, scalpels, and the like, as they are mounted and exchanged on the robotic arms. The cameras and other sensors will also track the patient anatomy and surgical personnel which are in close proximity to the robotic arms allowing the collision avoidance system to continuously observes changes in the positions of all objects in the surgical field so that the controller can manipulate the surgical robotic arms to avoid collision between the arms (including their tools and end effectors) with other objects in the surgical field.

In some embodiments at least one of the arms of the surgical robot comprises a surveillance arm that carriers a camera or other sensor but which does not otherwise participate in the surgical procedure. The cameras or other sensors gather information about the constantly changing environment in the surgical space and deliver that information to the common controller. In preferred embodiments, the common controller will be configured to position and reposition the surveillance arm(s) to allow the cameras or other sensors to better visualize or sense the positions of the objects in the surgical space. In addition to positioning the surveillance arm(s), the common controller will usually be able to move and redirect the camera or other senor relative to the surveillance arm to observe specific regions of the surgical space.

In one aspect, the present invention provides a surgical robotic collision avoidance system comprising a surgical robot including at least one surveillance arm and at least one and usually at least two surgical arms. The at least one surveillance arm and the at least two surgical arms are mounted on a chassis that defines a surgical workspace. A camera or other optical sensor may be mounted on the at least one surveillance arm, and the at least two surgical arms are typically configured to hold and manipulate robotic surgical tools, cannulas, and the like, referred to collectively as “end effectors,” in a fixed or adjustable position relative to a distal end of the surgical arm. The surgical robot further includes a controller which is typically configured to (a) kinematically position the at least two surgical arms within the surgical workspace to perform a procedure on a patient, (b) kinematically or otherwise position the at least one surveillance arm to orient the camera optically observe a position of the patient and/or surgical personnel during the procedure, and (c) kinematically reposition one or both of the at least two surgical arms as necessary to avoid collisions with the patient or the surgical personnel based on the optically observed position(s) of the patient anatomy and/or the surgical personnel.

While the controller will kinematically position and track the surgical and surveillance arms of the robot, the positions of the patient anatomy, surgical personnel, and tools and other objects not attached to the surgical arms will typically be tracked by the camera or the sensors. As the position of the camera/sensor itself is kinematically tracked, however, the positions of the other objects observed by the camera/sensors can be determined by the controlled based on the known position of the camera/sensor in the surgical field and the visualized or sensed locations of the other objects in the field of view or the camera or a sensed position by the sensor.

“Kinematic” positioning and repositioning of the surveillance arm and surgical robotic arms within the surgical space means that the controller positions the distal ends of the arms primarily or solely based upon the dimensions, geometries, and of the robotic arms without the need to optically or otherwise track movement of the distal end. For example, in the typical case of articulated robotic arms, the controller can calculate and track the position of the distal end of each arm based upon the dimensions of each component or link of the arm and the direction and degree of bending between adjacent components. Such kinematic tracking of the robotic arms may be well known in the art of surgical and other robots and needs no further description.

In addition to kinematic positioning and repositioning of the surgical robotic arms, the controller will typically also kinematically track the positions of all or specific portions of the end effectors which may be attached to the distal ends of the surgical robot arms. For example, the dimensions and attachment details of the end effectors may be uploaded to the controller. Alternatively, the controller can determine the orientations of the end effectors as described in WO2023/144602, entitled “Intraoperative Robotic Calibration and Sizing of Surgical Tools PCT/IB2022/058978,” and commonly owned herewith, the full disclosure of which may be incorporated herein by reference.

“Optical” tracking of the position of the patient and/or surgical personnel will be accomplished by the camera or other optical sensor mounted on the surveillance arm. The position of the patient will typically be registered in the surgical space based upon the position of a fiducial or other marked affixed to the patient anatomy. As the position of the camera or other sensor will be kinematically tracked by the controller, the position of the fiducial and thus of the patient will also be known in the surgical space. Typically, a model of the relevant patient anatomy will be determined by scanning with the camera at the outset of the procedure.

The phrase “surgical space” as used herein refers to the space surrounding the patient undergoing a surgical procedure. It may be expected that the surveillance arm and surgical robotic arms will be positioned primarily or entirely within the surgical space with the positions of the arms including their distal ends being kinematically tracked by the controller over time. While the present invention does not exclude further optical tracking of the robotic arms (or portions thereof), such additional tracking will normally not be necessary and tracking will typically be accomplished solely by kinematic techniques.

The robotic arms will typically be positioned initially and the beginning of a procedure and will subsequently be repositioned over time during the procedure as required by the surgical protocol as well as to avoid collisions this the patient anatomy, surgical personnel, and with each other. While collision avoidance among the surgical arms can typically be accomplished with reliance solely on the kinematically tracked positions of the robot arms, collisions between the robotic arms and the patient anatomy and/or surgical personnel will be accomplished based on a combination of optical tracking of the locations of the patient anatomy and surgical personnel and kinematic tracking of the robotic arms.

In specific instances, the controller may be configured to orient the camera to optically observe a one or more anatomical markers on the patient anatomy, whereby the controller can calculate changes in patient position in real time. Typically, the anatomical markers are fiducials affixed to the patient.

In specific instances, the controller may be configured to scan the patient with the camera prior to the surgical procedure to provide an anatomical model of the patient.

In specific instances, the controller may be configured to kinematically reposition either or both of the at least two surgical arms within the surgical workspace to avoid collisions with each other based on kinematically tracked positions of the at least two surgical arms within the surgical workspace.

In specific instances, the controller may be further configured to reposition the at least two surgical arms within the surgical workspace without reference to optical information from the camera.

In specific instances, the chassis comprises a mobile chassis configured to be deployed adjacent a patient during surgery. For example, the chassis may consist essentially of a single structure. Alternatively, the chassis may comprise two or more component structures that may be fixedly joined.

In specific instances, the controller may be further configured to position and reposition the third surgical robotic arm automatically to orient and reorient the camera.

In specific instances, the controller may be further configured to allow a user to manually position and reposition the third surgical robotic arm to orient and reorient the camera.

In another aspect, the present invention provides a method for avoiding collision avoidance during a robotic surgical procedure. The method comprises kinematically controlling the movement of a first robotic surgical arm in a surgical space, kinematically controlling the movement of a second robotic surgical arm in the surgical space, and optically tracking the position(s) of a patient anatomy and/or surgical personnel in the surgical space with a camera held by a third robotic surgical arm. The third surgical robotic arm may be positioned and repositioned to orient and reorient the camera to observe the position(s) of the patient anatomy and/or the surgical personnel as said positions may change in the surgical space over time. Kinematically controlling movements of the robotic surgical arms in the surgical space comprises adjusting said movements to avoid collisions between said arms and the patient and/or the surgical personnel based on (a) the optically observed position(s) of the patient anatomy and/or the surgical personnel and (b) the kinematically tracked positions of the robotic surgical arms.

In specific instances, kinematically controlling the movements of the first and/or robotic surgical arms in the surgical space further comprises adjusting said movements to avoid collisions among said arms based solely on the kinematically tracked positions of said arms.

In specific instances, optically tracking the position(s) of a patient anatomy and/or surgical personnel in the surgical space with a camera held by a third robotic surgical arm comprises observing a marker affixed to the patient anatomy.

In specific instances, the method of the present invention further comprises using the third surgical robot arm to scan the patient anatomy with the camera to generate a model of the patient anatomy prior to the surgical procedure, wherein the optically observed positions of the patient anatomy are based upon the model of the patient anatomy.

In specific instances, positioning and repositioning the third surgical robotic arm to orient and reorient the camera may be automatically performed by the system.

In specific instances, positioning and repositioning the third surgical robotic arm to orient and reorient the camera may be selectively performed by a user.

In specific instances, the third surgical robotic arm may be kinematically positioned and repositioned to orient and reorient the camera.

In specific instances, the third surgical robotic arm may be positioned and repositioned to orient and reorient the camera based upon the image generated by the camera.

### BRIEF DESCRIPTION OF THE DRAWINGS

FIG. 1 shows a surgical robot with a collision avoidance system according to an embodiment of the present invention.

FIG. 2 shows the collision avoidance features of FIG. 1 operating to detect and prevent a potential collision between two robotic arms according to an embodiment of the present invention.

FIG. 3 shows the collision avoidance features of FIG. 1 operating to detect and prevent a potential collision between a robotic arm and a surgeon according to an embodiment of the present invention.

FIG. 4 is a chart setting forth the steps in an exemplary method of the disclosed technology.

### DETAILED DESCRIPTION OF THE INVENTION

With reference now to the figures and several representative embodiments of the invention, the following detailed description is provided. The following novel invention describes a system and method to prevent collisions actively and dynamically in a multi-arm surgical robotic system.

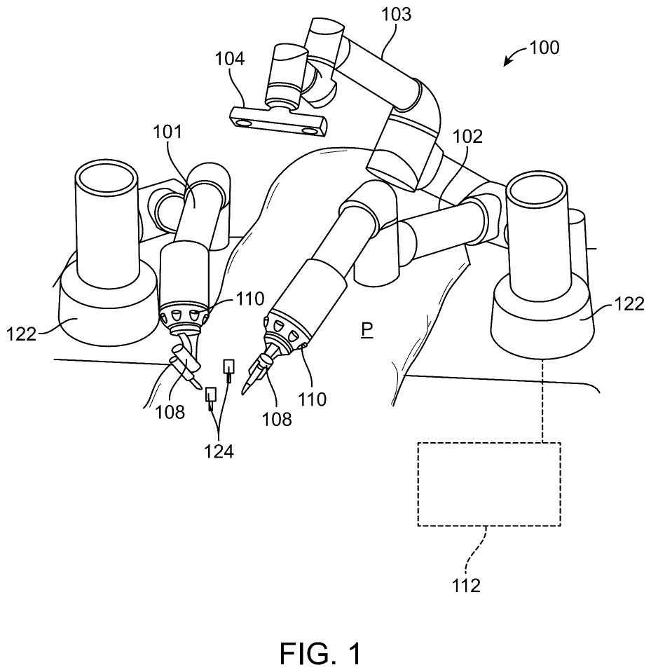

In one embodiment of the present invention, as shown in FIG. 1, a surgical multi-arm robot 100 comprises a collision avoidance system in accordance with the principles of the present invention. The robot 100 typically comprises at least two robotic arms 101 and 102 which are mounted on a common chassis, cart or other base chassis 120 and which are controlled by a controller 112. Robotic arms 101 and 102 are dedicated to surgical activities and a third, surveillance arm 103 carries a camera or sensor 104. As described thus far, the system could be constructed as described in commonly owned PCT publication WO2022/195460, the full disclosure of which is incorporated herein by reference.

Unlike the robot described in WO2022/195460, the controllers of the surgical robots disclosed herein will be configured to utilize the camera or other sensors of the surgical robot to track other objects in the surgical field and determine whether such objects are at risk of colliding or otherwise interfering with the surgical robotic arms as the arms are being manipulated by the controller in the surgical field.

For example, robotic controller 112 will be initially uploaded with the shapes, dimensions, and other physical characteristics of the robotic arms 101, 102, and 103 prior to the surgical procedure. In this way, the controller can kinematically position, reposition, and track all portions of the robot arms during the procedure, particularly including the distal ends 110 of the arms with carry the tools 108 and other end effectors. The shapes, dimensions, and other physical characteristics of the tools and other end effectors will also be provided, and the controller can then detect when particular tools have been mounted on each of the robotic arms. Thus, at all times, the controller will be able to kinematically track the positions of the arms, tools, and end effectors during the surgical procedure. Optical or sensor tracking is unnecessary (although not excluded) other than to observe what tools have been attached to the surgical arm. Alternatively, or additionally, the tools may be encoded or otherwise marked to allow the controller to identify the tool or end effector without optical or sensor-based identification.

The surveillance arm 103 will typically not participate in the surgical procedure other than to allow observation and will thus be free to continuously monitor the surgical surroundings, including being repositioned to better observe specific portions of the surgical space. The surveillance arm 103 can hold more than one camera/sensor in order to provide several layers of diverse data, and multiple arms/sensors can provide data from different angles or data of different types, and multiple surveillance arms can be achieved to provide different perspectives.

This surveillance arm 103 and camera/sensors 104 can scan and map the surface of the patient including bone markers 124 and surroundings at the beginning of the surgery, as is known in the prior art, to provide a surface map of the patient. As the procedure is performed, the camera/sensors 104 will can frequently or continuously track the markers 124 to update the position of the patient surface map in the surgical space since the patient position and surgical environment can change over time. Prior art cameras are usually employed in performing the surgery so cannot be relied upon for collision detection. In the present inventive system, since the surveillance arm is typically dedicated to collision detection, the system can continuously scan the patient and update the surface map with new information, such as for example that now there is a surgical tool in the patient's body and this area needs to be avoided.

The surveillance arm 103 arm can carry more than one camera/sensor 104 and thus provide additional information. For example, one surveillance arm can carry a navigation camera/sensor and continuously detect navigation markers that are placed on the robotic arms (not shown) or on the patient anatomy. This diversity of information can enhance the robustness of the data collected and can help facilitate a safer environment. Additionally, an additional surveillance arm can hold and carry sensors that can communicate with other sensors that are embedded in the other surgical robotic arms, surgical table etc. the main advantage is that this arm is free for this task from any surgical task.

Such multi-arm synchronization to achieve collision avoidance will have additional benefits. The central controller can actively and dynamically choose an optimal position to position the surveillance arm and improve the probability to detect a possible collision. For example, if the surgeon is using one of the arms for a particular task, the controller will know that and will be able to position the surveillance camera in an optimal location to detect a collision with the patient anatomy, the surgeon, or other objects introduced into the surgical space that would not be kinematically tracked by the controller, e.g., loose tools set down in the surgical space by the surgeon. Also, by proper positioning the sensors on the surveillance arm will have better chances to track the positions of the surgeon's hands to avoid contact. In preferred instances, the controller will use predictive algorithms to actively and dynamically position the surveillance arm and cameras/sensors in optimal locations in the surgical space wherein it will have higher probability to detect the possible collision.

While mobile robotic systems according to the present invention will preferably provide all apparatus and functions necessary to perform the collision detection protocols as described herein, the use of additional cameras, sensors, controllers, and the like, which are not part of the mobile system is not precluded. For example, pedestal-based and/or wall-mounted cameras and sensors may be used to assist the cameras and sensors on a mobile cart or chassis.

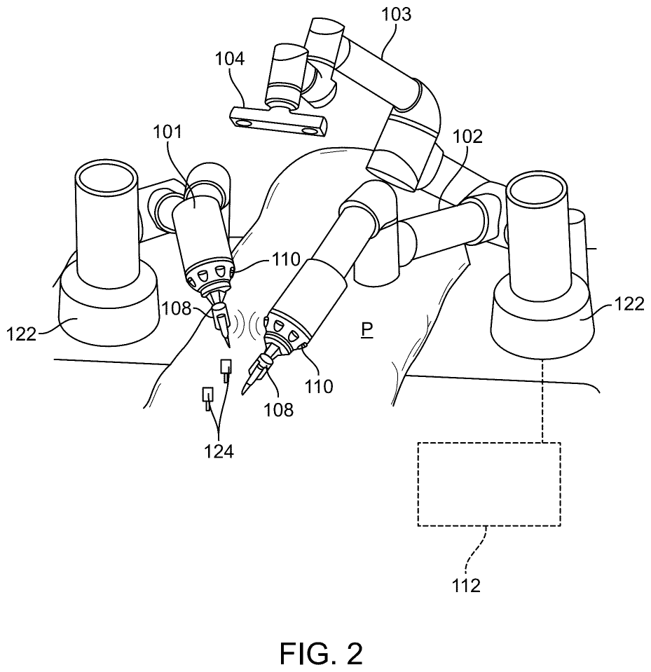

As shown in FIG. 2, the collision avoidance system of the present invention detects a potential collision between two robotic arms 201, 202. In most cases, the collision avoidance system relies solely on kinematic tracking of the arms 101 and 102 and tools 108, but in some instances optical or other sensor based tracking by the camera/sensors 104 may supplement or replace kinematic tracking. Reliance on kinematic tracking alone is possible because all arms of the surgical robot are being moved in a single coordinate space defined by a single chassis or cart so that arm/tool positions in space are continuously known to the controller. Multiple carts or chassis can be rigidly linked to provide a common and fixed coordinate space, as described in commonly owned PCT Patent Application PCT/IB2023/056911, entitled “Integrated Multi-Arm Mobile Surgical Robotic System,” the full disclosure of which is incorporated herein by reference.

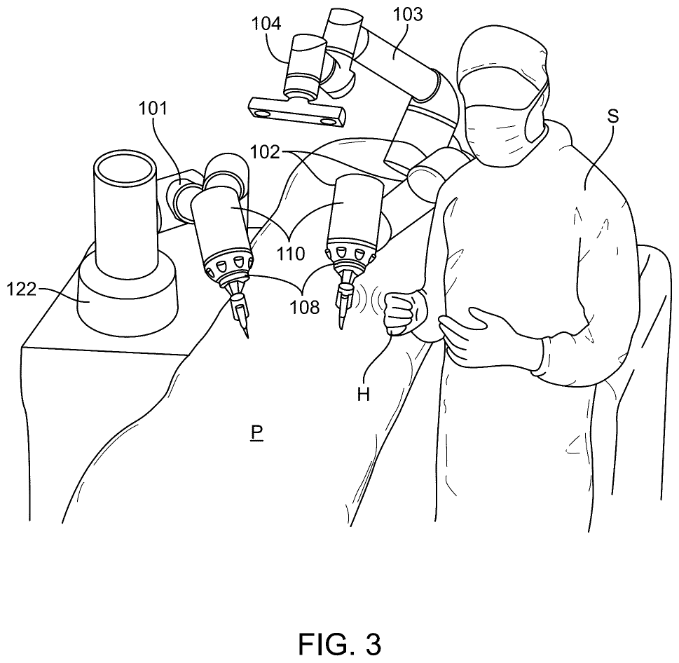

As shown in FIG. 3, the collision avoidance system of the present invention is used to avoid a collision between robotic arm 102 and a surgeon S. In this instance, the position of the surgical robotic arm 102 and tool 108 is kinematically tracked by the controller 112 without the need for optical or other tracking (although such additional tracking is not precluded). In contrast, the position of the surgeon, and in particular of the surgeon's hand H, is tracked by the camera/sensor 104.

This approach is advantageous, as the camera/sensor 104 need see only the patient markers 124 and the surgeon's hand H. The surveillance arm 103 can be moved as needed to position the camera/sensor 104 to optimize the view of the surgeon's hand h and the markers 124. S the surveillance arm is not being used in the surgery, it can be positioned and repositioned continuously or as often as necessary to monitor the surgical space as it is changing during the entire course of the surgical procedure.

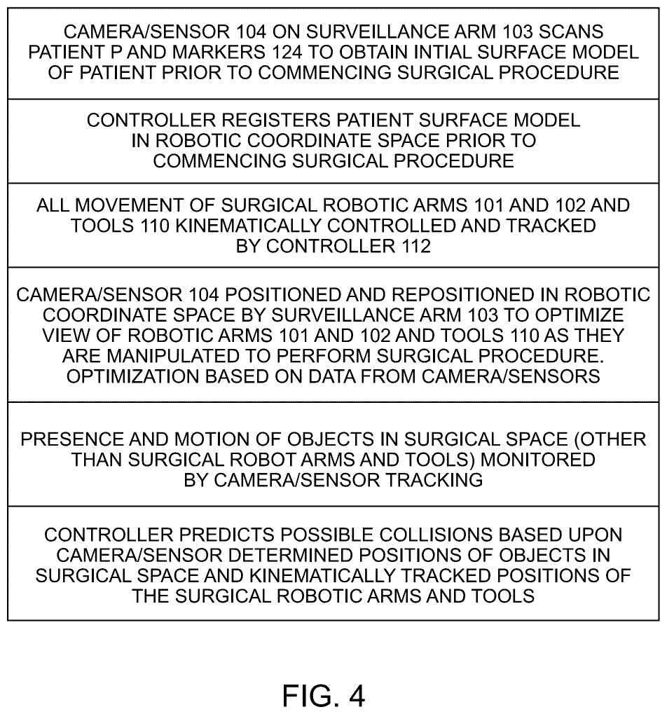

Methods as disclosed herein are summarized in the chart of FIG. 4. The camera/sensor 104 on surveillance arm 103 scans patient P and markers 124 to obtain a surface model of patient prior to commencing surgical procedure. The controller 112 registers the patient surface model in robotic coordinate space prior to commencing surgical procedure. Movement of surgical robotic arms 101 and 102 and tools 110 is kinematically controlled and tracked by controller 112. Camera/sensor 104 is positioned and repositioned in a robotic coordinate space by the surveillance arm 103 to optimize a view of robotic arms 101 and 102 and tools 110 as they are manipulated to perform surgical procedure. Optimization is based on data from camera/sensors. Presence and motion of objects in surgical space (other than surgical robot arms and tools) is monitored by camera/sensor tracking, and the controller 112 predicts possible collisions based upon camera/sensor determined positions of objects in surgical space and kinematically tracked positions of the surgical robotic arms and tools.

One of skill in the art will also realize that the embodiments provided herein are representative in nature. Departures from the provided embodiments that change, for example, the number and position of sensors or cameras on the surveillance arm, are within the scope and spirit of the present invention.

1. A surgical robotic collision avoidance system comprising:
a surgical robot comprising at least one surveillance arm and at least two surgical arms, wherein the at least one surveillance arm and the at least two surgical arms are mounted on a chassis that defines a surgical work space;
a camera or other sensor mounted on the at least one surveillance arm; and
a controller;
wherein the controller is configured to (a) kinematically position the at least two surgical arms within the surgical workspace to perform a procedure on a patient, (b) position the at least one surveillance arm to orient the camera to optically observe a position of the patient and/or surgical personnel during the procedure, and (c) kinematically reposition one or both of the at least two surgical arms as necessary to avoid collisions with the patient and/or the surgical personnel based on optically observed position(s) of the patient anatomy and/or the surgical personnel.

2. The system of claim 1, wherein the controller is further configured to orient the camera to optically observe one or more anatomical markers on the patient anatomy, whereby the controller can calculate changes in patient position in real time.

3. The system of claim 2, wherein the anatomical markers are fiducials affixed to the patient, preferably affixed to a bone of the patient.

4. The system of claim 1, wherein the controller is configured to scan the patient with the camera prior to the surgical procedure to provide an anatomical model of the patient.

5. The system of claim 1, wherein the controller is configured to kinematically reposition either or both of the at least two surgical arms within the surgical workspace to avoid collisions with each other based on kinematically tracked positions of the at least two surgical arms within the surgical workspace.

6. The system of claim 5, wherein the controller is further configured to reposition either or both of the at least two surgical arms within the surgical workspace without reference to optical information from the camera.

7. The system of claim 1, wherein the chassis comprises a mobile chassis configured to be deployed adjacent a patient during surgery.

8. The system of claim 7, wherein the chassis consists essentially of a single structure.

9. The system of claim 7 wherein the chassis comprises two or more component structures that may be fixedly joined.

10. The system of claim 1, wherein the controller is further configured to position and reposition a third surgical robotic arm automatically to orient and reorient the camera.

11. The system of claim 10, wherein the controller is further configured to allow a user to manually position and reposition the third surgical robotic arm to orient and reorient the camera.

12. A method of collision avoidance during a robotic surgical procedure, said method comprising:
kinematically controlling the movement of a first robotic surgical arm in a surgical space;
kinematically controlling the movement of a second robotic surgical arm in the surgical space;
optically tracking the position(s) of a patient anatomy and/or surgical personnel in the surgical space with a camera held by a third robotic surgical arm;
positioning and repositioning the third surgical robotic arm to orient and reorient the camera to observe the position(s) of the patient anatomy and/or the surgical personnel as said positions may change in the surgical space over time;
wherein kinematically controlling the movements of the robotic surgical arms in the surgical space comprises adjusting said movements to avoid collisions between said arms and the patient and/or the surgical personnel based on (a) the optically observed position(s) of the patient anatomy and/or the surgical personnel and (b) the kinematically tracked positions of the robotic surgical arms.

13. The method of claim 12, wherein kinematically controlling the movements of the first and/or robotic surgical arms in the surgical space further comprises adjusting said movements to avoid collisions among said arms based solely on the kinematically tracked positions of said arms.

14. The method of claim 12, wherein optically tracking the position(s) of a patient anatomy and/or surgical personnel in the surgical space with a camera held by a third robotic surgical arm comprises observing a marker affixed to the patient anatomy.

15. The method of claim 12, further comprising using the third surgical robot arm to scan the patient anatomy with the camera to generate a model of the patient anatomy prior to the surgical procedure, wherein the optically observed positions of the patient anatomy are based upon the model of the patient anatomy.

16. The method of claim 12, wherein positioning and repositioning the third surgical robotic arm to orient and reorient the camera is automatically performed by the system.

17. The method of claim 12, wherein positioning and repositioning the third surgical robotic arm to orient and reorient the camera is selectively performed by a user.

18. The method of claim 12, wherein the third surgical robotic arm is kinematically positioned and repositioned to orient and reorient the camera.

19. The method of claim 12, wherein the third surgical robotic arm is positioned and repositioned to orient and reorient the camera based upon the image generated by the camera.

20.-33. (canceled)
